(19) United States
(12) Patent Application Publication
(10) Pub. No.: US 2011/0190886 A1
(43) Pub. Date: Aug. 4, 2011

(54) BRAIDED TERTIARY NANOFIBROUS STRUCTURE FOR LIGAMENT, TENDON, AND MUSCLE TISSUE IMPLANT

(75) Inventor: Wan-Ju Li, Madison, WI (US)

(73) Assignee: WISCONSIN ALUMNI RESEARCH FOUNDATION, Madison, WI (US)

(21) Appl. No.: 12/696,245

(22) Filed: Jan. 29, 2010

(51) Int. Cl. A61F 2/08; B29D 99/00; B05D 3/00; C12N 5/071

(52) U.S. Cl. 623/13.19; 264/103; 427/2.24; 435/395

(57) ABSTRACT

A fabricated braided nanofibrous structure having nanofiber bundles that independently have nanofibers oriented in a uniaxial direction and methods of preparing braided nanofibrous structures. More specifically, the braided nanofibrous structures have at least three nanofiber bundles, wherein the nanofiber bundles are braided together and each nanofiber bundle independently includes at least two nanofibers oriented in a uniaxial direction. The braided nanofibrous structures may be inserted into a region of damaged ligament, tendon, or muscle in a subject.

### BACKGROUND OF THE DISCLOSURE

The present disclosure generally relates to braided nanofibrous structures. Particularly suitable structures are unique braided nanofibrous structures having enhanced cellular activities and extracellular matrix formation, whereby active mechanical stimuli and enhanced medium circulation in a bioreactor allow cell-laden braided nanofibrous structures to develop into physiologically functional ligament, tendon, and muscle tissue grafts. Of particular importance in this disclosure are fabricated tissue engineered braided nanofibrous structures with biologically favorable structural and mechanical properties for ligament, tendon, and muscle tissue graphs that may be transplanted to restore ligament, tendon, and muscle tissue functions.

Ligament, tendon, and muscle tissue injury frequently occurs in people actively participating in physical and recreational activities. In the United States, for example, there are 577,400 physician visits for acute cruciate ligament injury and approximately 150,000 anterior cruciate ligament reconstruction surgeries performed annually. Current clinical treatments include implantation of ligament and tendon prosthesis or transplantation of autologous or allogeneic ligament and tendon grafts. Artificial materials, such as GORE-TEX for example, have been used clinically, but suffer from the problem of an inability to sustain the mechanical properties required for ligament and tendon functions and the strength of the material significantly deteriorates after implantation, which leads to the eventual failure of the prosthesis. Alternatively, natural materials from cadavers or autologous or allogeneic grafts, for example, are commonly used as ligament and tendon grafts for replacement therapy. Natural materials, however, suffer from disadvantages such as the lack of a sufficient amount of safe and functional biological grafts available for transplantation, transmission of diseases, and eventual failure.

Engineered tissue provides another promising alternative for replacement of defective tissue in which a live, natural tissue/support composition is generated from a construct made from a subject's own cells in combination with a support composition. For quality ligament, tendon, and muscle tissue regeneration, cultured cells and newly formed tissues require an adequate amount of mechanical stimulation to encourage cell growth and facilitate extracellular matrix deposition, but must also be protected from extreme mechanical stresses during regeneration (ex vivo and in vivo) and transplantation. Engineered tissues, however, suffer from challenges such as, the lack of engineered structures having favorable biological structure and mechanical properties and the lack of mechanical stimulation protocols effective for ex vivo quality ligament, tendon, and muscle tissue regeneration. Thus, a mechanically sound structure that satisfies these requirements is needed for successful ligament, tendon, and muscle tissue engineering.

Accordingly, there is a need in the art for alternative approaches for treating damaged or defective ligament, tendon, and muscle tissue. Specifically, it would be advantageous to construct ligament, tendon, and muscle grafts having excellent mechanical properties and capable of carrying out highly demanding mechanical functions. These ligaments, tendons, and muscle tissue may be broadly applicable in degenerative diseases and ligament, tendon, and muscle tissue injury.

### SUMMARY OF THE DISCLOSURE

The present disclosure is generally directed to braided nanofibrous structures. More specifically, in one aspect, the present disclosure is directed to unique braided nanofibrous structures comprising at least three nanofiber bundles, wherein the nanofiber bundles are braided together and each nanofiber bundle independently comprises at least two nanofibers oriented in a uniaxial direction. In certain embodiments, the braided nanofibrous structures are used as ligament replacement structures. In other embodiments, the braided nanofibrous structures are used as tendon replacement structures. In still other embodiments, the braided nanofibrous structures are used as muscle tissue replacement structures.

In some embodiments, the present disclosure is directed to braided nanofibrous structures further comprising a plurality of cells.

In another aspect, the present disclosure is directed to methods of preparing a braided nanofibrous structure, the method comprising preparing a nanofiber bundle comprising at least two nanofibers oriented in a uniaxial direction, and preparing a braided nanofibrous structure by braiding at least three nanofiber bundles together.

In some embodiments, the method further comprises seeding the braided nanofibrous structure with a plurality of cells to form a braided nanofibrous structure having cells dispersed therein and culturing the braided nanofibrous structure having cells dispersed therein in a bioreactor.

In other embodiments, the method further comprises inserting the braided nanofibrous structure into a region of damaged tissue in a subject.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 shows an example of an electrospinning apparatus useful to form nanofibers.

FIG. 2A is a SEM image (1000×) showing an example of a nanofiber bundle showing nanofibers oriented in a uniaxial direction.

FIG. 2B is a SEM image (500×) showing an example of a nanofiber bundle showing nanofibers oriented in a uniaxial direction.

FIG. 3 is an illustration showing the hierarchal structure of a braided nanofibrous structure.

FIG. 4 is an illustration showing braided nanofibrous structures comprising three (A), four (B), and five (C) nanofiber bundles.

FIG. 5 shows macroscopic images of braided nanofibrous structures comprising three (A), four (B), or five (C) nanofiber bundles braided together.

FIG. 6 shows tensile load-displacement curves of poly(L-lactic acid) braided nanofibrous structures with 3, 4, or 5 nanofiber bundles.

FIG. 7 shows cell proliferation of human mesenchymal stem cells cultured in a three-nanofiber bundle braided nanofibrous structure after 7, 14, and 21 days in culture.

FIG. 8 is a SEM image of human mesenchymal stem cells cultured in a three-nanofiber bundle braided nanofibrous structure after 7 days.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein may be used in the practice or testing of the present disclosure, suitable methods and materials are described below.

### Braided Nanofibrous Structures

The present disclosure is generally directed to braided nanofibrous structures. More specifically, in one aspect, the present disclosure is directed to unique braided nanofibrous structures including at least three nanofiber bundles, wherein the nanofiber bundles are braided together and each nanofiber bundle independently includes nanofibers oriented in a uniaxial direction.

It has now been found that the braided nanofibrous structures of the present disclosure more closely mimic natural collagen fibers, promote directionality of cells growing on and/or in braided nanofibrous structures, and have improved mechanical properties. For example, cells grown on nanofibers oriented in a uniaxial direction are spindle-shaped and oriented in the nanofiber direction, whereas cells grown on randomly oriented nanofibers have no directionality. Further, it is believed that nanofiber alignment in a uniaxial direction promotes oriented cells to synthesize extracellular matrix (e.g., collagen).

Surprisingly, it has been found that fibers in the nanometer size range more closely mimic natural fibers and thus provide a biologically favorable structure for cellular activities than do other braided structures prepared using microfibers. A “nanofiber” refers to ultra-fine fibers having a diameter in the nano-sized range. Nanofibers have a particular diameter, depending on, for example, the type of material used to prepare the nanofiber, fabrication protocols, and the like. Suitable nanofiber diameter may be about 2 μm or less. In one embodiment, the nanofibers include a diameter of less than about 1 μm. Other suitable nanofiber diameter may be from about 50 nm to about 1000 nm. More suitably, the nanofibers include a diameter of from about 500 nm to about 900 nm. Nanofiber diameter can be determined, for example, by scanning electron microscopy.

In one aspect, the braided nanofibrous structures include nanofiber bundles including at least 90% nanofibers. More suitably, the braided nanofibrous structures include about 95% nanofibers. Even more suitably, the braided nanofibrous structures include about 96% nanofibers, about 97%, about 98%, or about 99% nanofibers. And even more suitably, the braided nanofibrous structures include 99% or more of nanofibers.

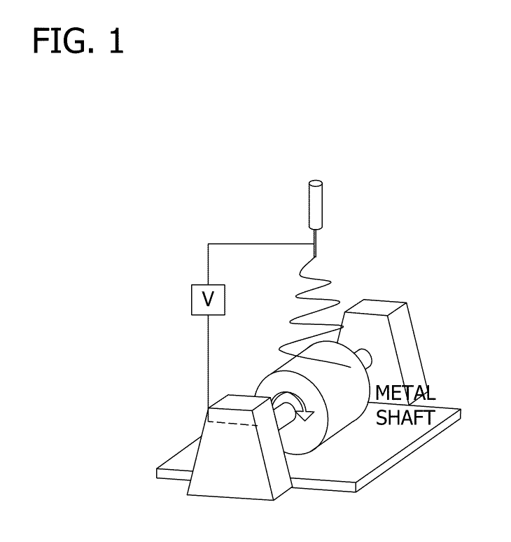

In one embodiment, the braided nanofibrous structures include nanofiber bundles wherein the individual nanofibers are electrospun nanofibers. Electrospun nanofibers may be made using an apparatus such as shown in FIG. 1. Unique physical characteristics of electrospun nanofibers enhance adsorption of cell adhesion molecules, induce favorable cell to extracellular matrix interactions, promote in vivo-like three-dimensional adhesion, activate cell signaling pathways, maintain cell phenotype, and support cell differentiation. Alternatively, nanofibers may be electro-blown nanofibers.

Nanofibers may be any fibrous material known in the art. Suitable nanofiber materials may include natural proteins and/or synthetic materials as known in the art. The nanofiber material may be biodegradable material and non-biodegradable material. Nanofibers may be any suitable synthetic material such as, for example, at least one of poly(glycolide) (PGA), poly(L-lactic acid) (PLA), poly(lactide-co-glycolide) (PLGA), poly(L-lactide) (PLLA), poly(D,L-lactide) (P(DLLA)), poly(ethylene glycol) (PEG), poly(ε-caprolactone) (PCL), montmorillonite (MMT), poly(L-lactide-co-ε-caprolactone) (P(LLA-CL)), poly(ε-caprolactone-co-ethyl ethylene phosphate) (P(CL-EEP)), poly[bis(p-methylphenoxy)phosphazene] (PNmPh), poly(3-hydroxybutyrate-co-3-hydroxyvalerate) (PHBV), poly(ester urethane) urea (PEUU), poly(p-dioxanone) (PPDO), polyurethane (PU), polyethylene terephthalate (PET), poly(ethylene-co-vinylacetate) (PEVA), poly(ethylene oxide) (PEO), poly(phosphazene), poly(ethylene-co-vinyl alcohol). Suitable natural proteins include, for example, at least one of collagen, elastin, hyaluronic acid, silk fibroin, gelatin, fibrinogen, chitin, chitosan.

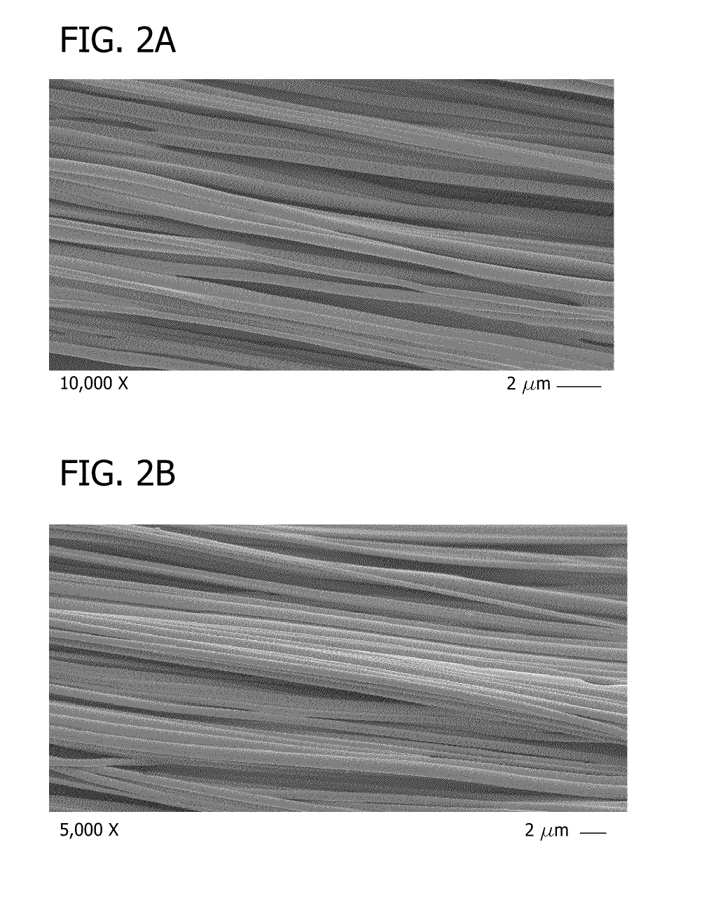

Nanofiber bundles of the braided nanofibrous structures include individual nanofibers that are aligned or oriented in a uniaxial direction. As used herein, “oriented in a uniaxial direction” refers to nanofiber bundles having a majority of individual nanofibers aligned in parallel. For example, greater than about 50% of the individual nanofibers of a nanofiber bundle are aligned parallel to other individual nanofibers of the nanofiber bundle. In another suitable embodiment, greater than about 60% of the individual fibers are aligned in parallel, more suitably, greater than about 70% of the individual fibers are aligned in parallel, even more suitably, greater than about 80%, even more suitably, greater than about 90%, even more suitably, greater than about 95%, even more suitably 99% or more of the individual fibers are aligned in parallel. FIG. 2A is a scanning electronmicrograph (SEM) of a nanofiber bundle showing individual nanofibers oriented in a uniaxial direction. Further, FIG. 2B is a SEM showing an example of a nanofiber bundle having nanofibers oriented in a uniaxial direction.

In one aspect, the nanofiber bundle is porous. Suitable nanofiber bundle porosity includes from about 10% to about 95% porosity. Suitable nanofiber bundle pore sizes may be from about 10 μm to about 800 μm. Pore size may be determined, for example, by scanning electron microscopy. Porosity and pore size of nanofiber bundles results in the also braided nanofibrous structure being porous. Porosity and pore size allow cells to migrate into and through pores and infiltrate into nanofiber bundles, allow culture medium circulation into and through pores, and allow exchange of nutrients and metabolic waste.

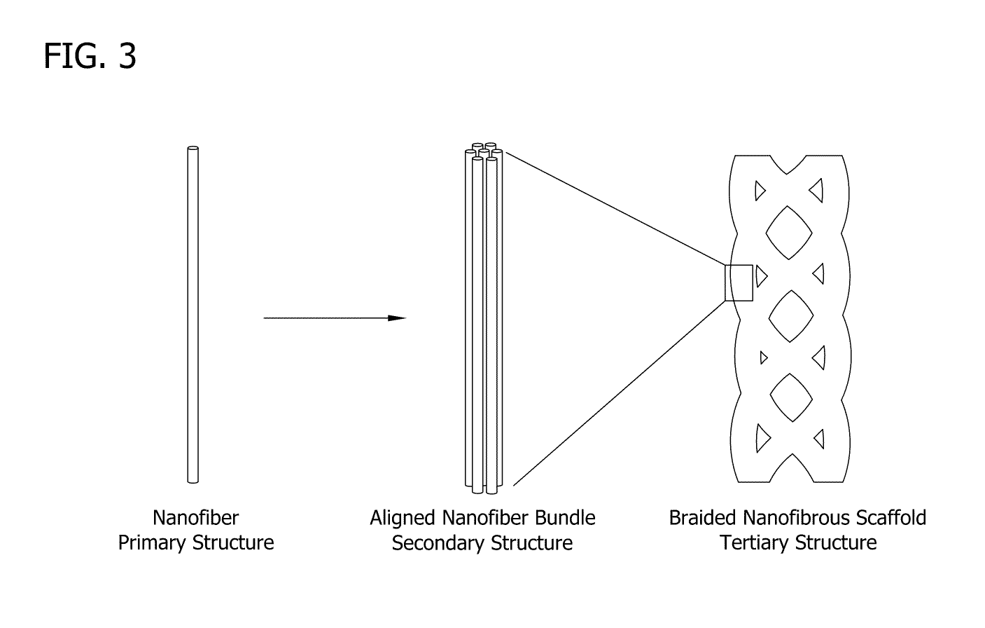

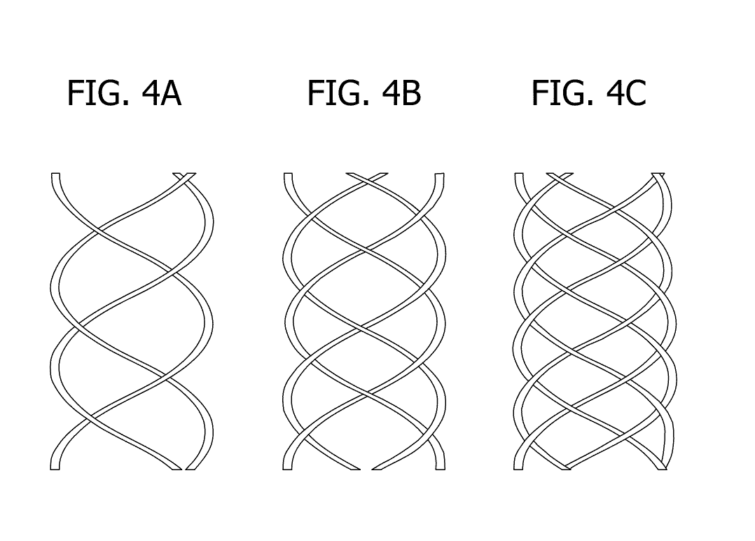

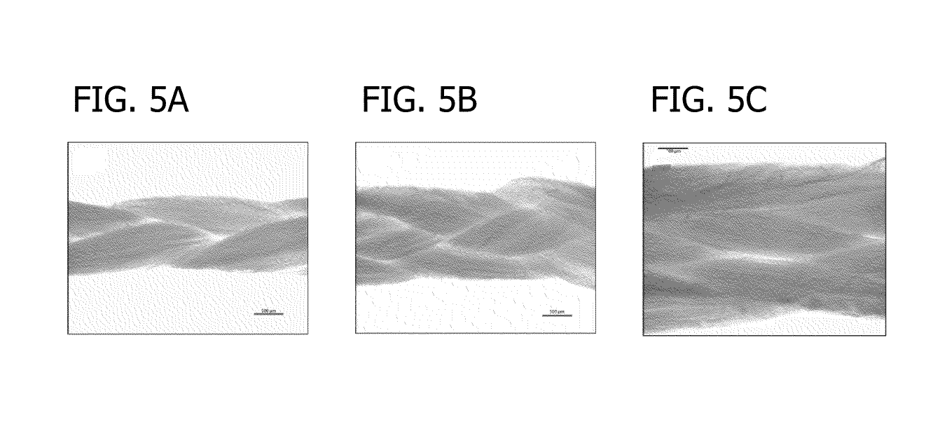

Braided nanofibrous structures include nanofiber bundles that are braided together. FIG. 3 is an illustration showing the hierarchal structure of braided nanofibrous structures. Individual nanofibers (i.e., Nanofiber Primary Structure) make up nanofiber bundles (i.e., Aligned Nanofiber Bundle Secondary Structure). Nanofiber bundles are braided to make up the braided nanofibrous structures (i.e., Braided Nanofibrous Scaffold Tertiary Structure). FIG. 4 is an illustration showing braiding patterns using three (A), four (B), or five (C) nanofiber bundles to form three-, four-, and five-nanofiber bundle braided nanofibrous structures. Further, FIGS. 5A, 5B, and 5C show the corresponding macroscopic images of the three-, four-, and five-nanofiber bundle braided nanofibrous structures.

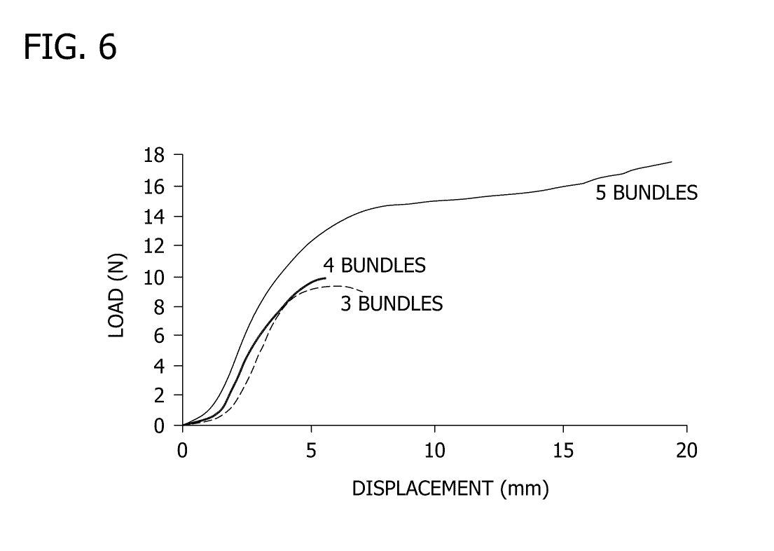

The braided nanofibrous structures including at least three nanofiber bundles of nanofibers oriented in a uniaxial direction have an improved strain, strength, and stiffness. For example, Table 1 shows maximal strain, maximal strength, and stiffness of PLLA braided nanofibrous structures with 3, 4, and 5 nanofiber bundles. A braided nanofibrous structure prepared for replacement of an anterior cruciate ligament prepared for insertion into a human subject, for example, may have a tensile strength of at least 1730 Newtons. Moreover, the number of nanofiber bundles that are braided together enhance strength, strain, and stiffness of the braided nanofibrous structure. FIG. 6 shows tensile load-displacement curves of PLLA braided nanofibrous structures with 3, 4, or 5 nanofiber bundles. Therefore, braided nanofibrous structures having varying desired mechanical properties can be achieved by varying the number of nanofiber bundles.

TABLE 1
Tensile properties of PLLA braided nanofibrous
structures of 3-, 4-, and 5-nanofiber bundles.

| | Max Strain | Max Strength | Stiffness |
|---|---|---|---|
| Bundles | (%) | (N) | (N/mm) |
| 3 | 11.3 ± 2.3 | 6.9 ± 1.4 | 2.31 ± 0.7 |
| 4 | 13.2 ± 1.8 | 7.8 ± 1.7 | 2.60 ± 0.1 |
| 5 | 24.1 ± 7.5 | 12.9 ± 2.8 | 3.36 ± 0.4 |

In another aspect, the braided nanofibrous structures include a plurality of cells. Cell types that may be used are, for example, ligament fibroblasts, tendon fibroblasts, muscle fibroblasts, muscle cells, mesenchymal stem cells, embryonic stem cells, and combinations thereof. The cells may be autologous cells, allogeneic cells, or xenogeneic cells. “Autologous cells” refers to cells that are donated and received by the same subject. For example, cells are obtained from subject A, incorporated into the braided nanofibrous structure, and the cell-laden braided nanofibrous structure is inserted into subject A. “Allogeneic cells” refers to cells that are donated by a subject that is different from the recipient subject, however, the donor subject and recipient subject are from the same species. For example, cells are obtained from subject A, incorporated into the braided nanofibrous structure, and the cell-laden braided nanofibrous structure is inserted into subject B. “Xenogeneic cells” refers to cells that are obtained from or donated by a species that is different than the recipient.

In another aspect, the braided nanofibrous structure may be a braided nanofibrous structure that is coated with a cell adhesion molecule. The cell adhesion molecule coating the braided nanofibrous structure contacts nanofibers making up the nanofiber bundles that, in turn, are braided together to prepare the braided nanofibrous structures. The cell adhesion molecule may be, for example, fibronectin, vitronectin, collagen, RGD peptide, laminin, and combinations thereof. Unique physical characteristics of electrospun or electro-blown nanofibers enhance adsorption of cell adhesion molecules, induce favorable cell to extracellular matrix interactions, promote in vivo-like three-dimensional adhesion and activate cell signaling pathways, maintain cell phenotype, and support cell differentiation.

### Methods of Preparing Braided Nanofibrous Structures

Another aspect of the present disclosure is directed to methods of preparing braided nanofibrous structures. In one embodiment, the method of preparing a braided nanofibrous structure includes preparing a nanofiber bundle including at least two nanofibers, wherein each nanofiber bundle independently includes at least two nanofibers oriented in a uniaxial direction and preparing a braided nanofibrous structure by braiding at least three nanofiber bundles together.

In another aspect, the method includes preparing a nanofiber bundle including at least two nanofibers, wherein each nanofiber bundle independently includes at least two nanofibers oriented in a uniaxial direction and preparing a braided nanofibrous structure by braiding at least three nanofiber bundles together, and further includes seeding the braided nanofibrous structure with a plurality of cells to form a braided nanofibrous structure having cells dispersed therein, and culturing the braided nanofibrous structure having cells dispersed therein in a bioreactor.

In one aspect of the method of preparing braided nanofibrous structures, the electrospinning method is used to form nanofibers as disclosed in Li et al. (J. Biomech. 40:1686-1693 (2007)). An apparatus such as that shown in FIG. 1 may be used for electrospinning. Briefly, a fiber material is prepared and electrospun into aligned or uniaxially oriented nanofibers in an electrospinning apparatus using the fabrication parameters such as that described in Li et al. (J. Biomech. 40:1686-1693 (2007)), which is hereby incorporated by reference to the extent that it is consistent herewith. Another alternative is to prepare nanofibers by electro-blowing. Material used to prepare nanofibers may be purchased from a commercial vendor (for example, Polysciences Inc., Warrington, Pa.).

Different materials, as described above, may be used to prepare nanofibers that are used in the method to prepare a nanofiber bundle. Furthermore, typically, as noted above, nanofibers include varying diameters depending on the type of material used to prepare the nanofiber, fabrication protocols, and the like. As more fully described above, the nanofibers include a diameter of about 2 μm or less. In another aspect, the nanofibers include a diameter of less than about 1 μm. In still another aspect, the nanofibers include a diameter from about 50 nm to about 1000 nm. In still another aspect, the nanofibers include a diameter from about 500 nm to about 900 nm. Nanofiber diameter can be determined, for example, by scanning electron microscopy. Individual nanofiber bundles include at least two individual nanofibers. Suitably, nanofiber bundles include two to 10,000 individual nanofibers. In other aspects, nanofiber bundles include greater than 10,000 individual nanofibers.

As prepared using electrospinning, the nanofibers are assembled in a uniaxial direction and may be assembled into nanofiber bundles as described above. The nanofiber bundles are then braided to form the braided nanofibrous structures. In one aspect, the braided nanofibrous structures are prepared by braiding at least three nanofiber bundles together. Braiding may be accomplished manually (i.e., by hand) or using a machine. To braid nanofiber bundles together to prepare braided nanofibrous structures, an individual nanofiber bundle is interwoven, twisted, or wound together with other nanofiber bundles in an overlapping pattern. See e.g., FIGS. 4 and 5.

The braided nanofibrous structures may be prepared with specific mechanical properties desired for the intended use. Mechanical properties that may be desired include, for example, the strain, strength, stiffness, flexibility, compliance, and/or other mechanical properties. The material used to prepare individual nanofibers may affect the mechanical properties of the nanofiber bundles and/or the braided nanofibrous structures. For example, if the material used to prepare individual nanofibers has high strain, strength, and/or stiffness, the nanofiber bundles and/or braided nanofibrous structures prepared from these individual nanofibers may also possess high strain, strength, and/or stiffness. Adjusting the diameter of individual nanofibers, as described in more detail above, may also affect the mechanical properties. For, example, braided fibrous structures prepared from small diameter nanofibers may be used to prepare strong braided nanofibrous structures. Alternatively, mechanical properties of the braided nanofibrous structures can be varied by adjusting the number of individual nanofibers used to prepare the nanofiber bundles braided together to prepare the braided nanofibrous structures. Further, the number of nanofiber bundles used to prepare the braided nanofibrous structures can affect mechanical properties of the braided nanofibrous structures.

As noted above, braided nanofibrous structures may include at least three nanofiber bundles braided together to prepare the braided nanofibrous structures. In some aspects, braided nanofibrous structures include three nanofiber bundles that are braided together to form braided nanofibrous structures. FIG. 5A is a macroscopic image of braided nanofibrous structure comprising three nanofiber bundles that are braided together to form a braided nanofibrous structure. In other aspects, braided nanofibrous structures include four nanofiber bundles that are braided together to form a braided nanofibrous structure. FIG. 5B is a macroscopic image of braided nanofibrous structure comprising four nanofiber bundles that are braided together to form a braided nanofibrous structure. In still other aspects, braided nanofibrous structures include five nanofiber bundles that are braided together to form braided nanofibrous structures. FIG. 5C is a macroscopic image of braided nanofibrous structure comprising five nanofiber bundles that are braided together to form a braided nanofibrous structure. The number of nanofiber bundles that are braided together may be varied depending on the desired strength, strain, stiffness, and/or other mechanical properties of the braided nanofibrous structure. See e.g., Table 1 and FIG. 6.

In one aspect, the method further includes seeding the braided nanofibrous structure with a plurality of cells to form a braided nanofibrous structure having cells dispersed therein and culturing the braided nanofibrous structure having cells dispersed therein in a bioreactor. “Seeding” the braided nanofibrous structure with a plurality of cells refers to dispersing the plurality of cells onto, along, down, against, in contact with, in proximity or the like, such that the plurality of cells contact the braided nanofibrous structure. Culturing the braided nanofibrous structure having cells dispersed therein in a bioreactor is intended to refer to conditions in which the braided nanofibrous structure provides a substrate on and/or in which the plurality of cells attach, proliferate, grow, and perform other cellular functions. Cellular braided nanofibrous structures and cell-laden braided nanofibrous structures are used herein interchangeably to refer to braided nanofibrous structures having cells dispersed therein.

Cell types that may be used are described above and may be, for example, ligament fibroblasts, tendon fibroblasts, muscle fibroblasts, muscle cells, mesenchymal stem cells, embryonic stem cells, and combinations thereof. Mesenchymal stem cells may be isolated from various tissues, including but not limited to, blood, bone marrow, cord blood, placenta, muscle, fat, and other tissues containing mesenchymal stem cells. The cells may be autologous cells, allogeneic cells, or xenogeneic cells.

To obtain cells for seeding, a tissue source is first obtained. Cells may be dissociated from the tissue by enzyme treatment using standard methods. For example, the tissue may be subjected to trypsin treatment or trypsin-collagenase treatment. Following enzyme treatment, the solution is subjected to centrifugation, for example, to remove contaminants and cellular clumps. The supernatant may then be subjected to a second centrifugation step to pellet individual cells. The cellular pellet is then resuspended in culture medium and cells are plated at the desired cell density. Alternatively, the intact tissue source may be placed in a culture dish to allow individual cells to migrate away from the tissue sample. Following a desired period in culture, for example 1 to 3 days, the cultured cells are subjected to an enzyme treatment to dissociate cultured cells from the culture vessel. Cells dissociated from the culture vessel are pelleted by centrifugation, for example, and the cells are resuspended in culture medium to obtain a desired cell density. The cells are then seeded onto pre-formed braided nanofibrous structures at a desired cell density. Seeded braided nanofibrous structures are then cultured in a bioreactor for a sufficient time to allow cells to attach, migrate, secrete extracellular matrix, secrete growth factors, proliferate, and exhibit other cellular activities. Suitable culture periods may be from less than about 1 day to about 21 days or more. As with other cell culturing methods, culturing refers to its ordinary meaning as understood by one skilled in the art to refer to the process by which cells are grown under controlled conditions such as, for example, temperature, CO₂ atmosphere, humidity, culture medium, and the like. Bioreactor is used according to its ordinary meaning as used in the art to refer to any device or system that supports a biologically active environment for growing cells or tissues.

The cell-laden or cellular braided nanofibrous structure is mechanically stimulated while in culture. For example, cell-laden or cellular braided nanofibrous structures may undergo cycles of tension followed by no tension. The cyclic tensile strain may be about 1% to about 30%. The frequency of the cyclic tensile strain may be about 0.01 Hz to about 20 Hz. Additionally or alternatively, the cell-laden or cellular braided nanofibrous structure may be subjected to culture medium circulation. Circulation of the culture medium may be accomplished by any method known in the art. For example, the culture medium may be exchanged or the culture medium may be circulated into and out of the bioreactor. Circulation of the culture medium also includes flow of the culture medium through pores of the nanofiber bundles.

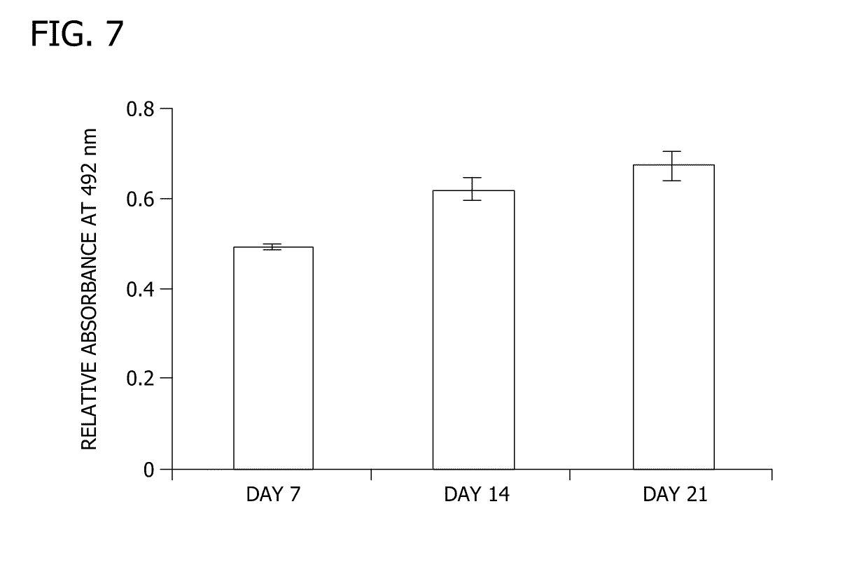

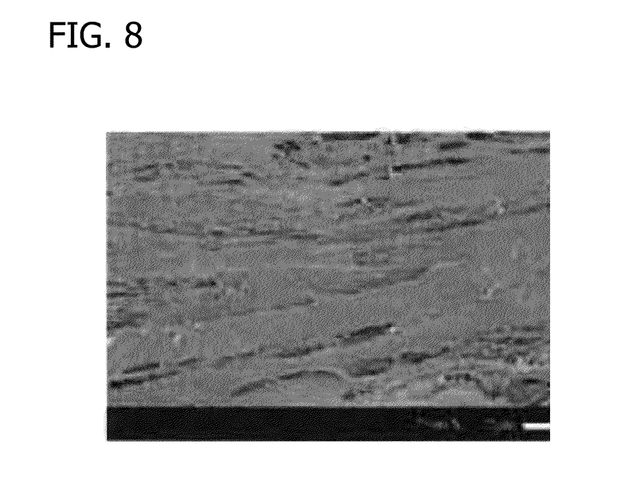

It is further intended that cells may migrate along nanofibers and/or into the pores of the nanofiber bundles. The cells may, for example, change shape, form cell-cell contacts, form cell-substrate contacts, synthesize, for example, collagen or other extracellular matrix molecules, activate cell signaling pathways, and/or differentiate while being cultured in the bioreactor. For example, FIG. 7 shows cell proliferation of human mesenchymal stem cells cultured in a three-nanofiber bundle braided nanofibrous structure for 7, 14, and 21 days. FIG. 8 is a SEM image of human mesenchymal stem cells cultured in a three-nanofiber bundle braided nanofibrous structure for 7 days.

In another aspect, the method further includes culturing the braided nanofibrous structure seeded with the plurality of cells in the presence of at least one growth factor as known in the art. The growth factor may be, for example, growth differentiation factors (GDFs), epidermal growth factors (EGFs), basic fibroblast growth factors (bFGFs), and transforming growth factor-beta (TGF-βs).

In another aspect, the method further includes coating the braided nanofibrous structure with at least one of a cell adhesion molecule and other bioactive molecule. In one embodiment, the cell adhesion molecule or other bioactive molecule coating the braided nanofibrous structure contacts nanofibers making up the nanofiber bundles that are then braided together to prepare the braided nanofibrous structures. In the method, nanofibers may be coated with the cell adhesion molecule or other bioactive molecule before nanofiber bundles are prepared. Alternatively or additionally, the nanofiber bundles may be coated with the cell adhesion molecule or other bioactive molecule before braiding the cell adhesion molecule-coated nanofiber bundles together. Alternatively or additionally, the braided nanofibrous structure is prepared and then coated with the cell adhesion molecule or other bioactive molecule. The cell adhesion molecule or other bioactive molecule may be applied by any method known in the art such as, for example, dipping, pipetting, spraying, soaking, or the like. The cell adhesion molecule may be, for example, at least one of fibronectin, vitronectin, collagen, RGD peptide, and laminin. Bioactive molecules may be, for example, peptides or proteins that enhance cellular activities such as, proliferation, differentiation, and matrix production such as, for example, cytokines.

The method may further include inserting the braided nanofibrous structure into a region of damaged tissue into a subject. The braided nanofibrous structure inserted may be a braided nanofibrous structure with or without cells. For example, a braided nanofibrous structure may be prepared according to the method in which nanofiber bundles are braided together to prepare a braided nanofibrous structure. This braided structure is then inserted into the subject. In another aspect, a cellular braided nanofibrous structure may be prepared according to the method in which nanofiber bundles are braided together to prepare a braided nanofibrous structure, seeded with cells, and cultured according to the method. The cellular braided nanofibrous structure is inserted into a region of damaged tissue into a subject. The braided nanofibrous structure inserted into a region of damaged tissue into a subject may be a braided nanofibrous structure that is or is not coated with a cell adhesion molecule or other bioactive molecule.

The subject may be any animal. For example, the animal may be a mammal such as, for example, a human, dog, cat, horse, pig, cow, rabbit, mouse, rat, hamster, sheep, and goat. The damaged tissue may be, for example, a ligament, a tendon, or muscle tissue. The damaged ligament or tendon may be, for example, a knee, shoulder, ankle, hip, wrist or other ligament or tendon. For example, the damaged ligament may be, for example, an anterior cruciate ligament, a posterior cruciate ligament, lateral collateral ligament, medial collateral ligament, and other ligaments. The damaged tendon may be, for example, patellar tendon, quadriceps tendon, rotator cuff, Achilles tendon, and other tendons. The muscle tissue may be, for example, biceps, triceps, deltoid, trapezius, pectoralis, quadriceps, hamstring, calf, forearm, and other muscle tissues.

The disclosure will be more fully understood upon consideration of the following non-limiting Examples.

### Example 1

In this Example, braided nanofibrous structures were prepared. Mechanical properties of the braided nanofibrous structures were then analyzed. Additionally, human mesenchymal stem cells were seeded onto braided nanofibrous structures and cell proliferation was analyzed.

### Electrospinning and Braided Nanofibrous Structure Preparation

Individual nanofibers used to create bundles that were subsequently used to prepare braided nanofibrous structures were electrospun from a solution containing 20 w/v % poly(1-lactic acid) (PLLA) in chloroform. The polymer solution was loaded into a 10-mL syringe connected to an 18-gauge blunt-tip needle and electrospun at 15 kV onto a rotating mandrel placed 20 cm from the needle tip and operated at a linear velocity of 9.0 m/s. Individual nanofibers collected from the mandrel were bundled together to form nanofiber bundles. Nanofiber bundles were then braided to create braided nanofibrous structures having 3, 4, or 5 nanofiber bundles made from electrospun nanofibers. Each braided nanofibrous structure, independent of the number of nanofiber bundles, was fabricated to have a weight of approximately 12.5 mg. Each nanofiber bundle of a 3-nanofiber bundle braided nanofibrous structure was 4.2 mg. Each nanofiber bundle of a 4-nanofiber bundle braided nanofibrous structure was 3.1 mg. Each nanofiber bundle of a 3-nanofiber bundle braided nanofibrous structure was 2.5 mg.

### Mechanical Properties of Braided Nanofibrous Structures.

Mechanical properties of braided nanofibrous structures were analyzed by applying a uniaxial tensile load onto the structure until the structure failed. Braided nanofibrous structures with average dimensions of 44 mm×2.07 mm×0.62 mm were loaded into a servohydraulic mechanical testing system (Bionix, 858; MTS, Minneapolis, Minn.) using a clamping system and 500 lb load cell (Eaton Corporation). Braided nanofibrous structures were preconditioned using 10 cycles of 1% strain. Braided nanofibrous structures were then loaded with the applied force in a direction parallel to fiber alignment to create a displacement rate of 5 mm/minute until failure. Maximum tensile strain, maximal tensile strength, and primary stiffness modulus from the linear portion of the load-displacement curve were analyzed. As summarized in Table 1, braided nanofibrous structures having 3 nanofiber bundles had a maximal strain of 11.3±2.3%, a maximal strength of 6.9±1.4 N, and stiffness of 2.31±0.7 N/mm. Braided nanofibrous structures having 4 nanofiber bundles had a maximal strain of 13.2±1.8%, a maximal strength of 7.8±1.7 N, and stiffness of 2.6±0.1 N/mm. Braided nanofibrous structures having 5 nanofiber bundles had a maximal strain of 24.1±7.5%, a maximal strength of 12.9±2.8 N, and stiffness of 3.36±0.4 N/mm.

### Cell Culture

Human mesenchymal stem cells were isolated from the femoral head of a patient undergoing total hip arthroplasty. The cells were cultured and expanded in Dulbecco's Modified Eagle's medium (DMEM) containing 10% fetal bovine serum, 0.2 mM ascorbic acid, and 100 U/mL penicillin/streptomycin. Cells of the fifth passage were seeded onto 2-cm long, 3-nanofiber bundle braided nanofibrous structures to a final cell density of 10×10⁶ cells/cm³. Cellular braided nanofibrous structures were harvested after 7, 14, and 21 days (n=3) for cell proliferation analysis using a MTS [3-(4,5-dimethylthaizol-2-yl)-5-(3-carboxymethoxyphenyl)-2-(4-sulfophynyl)-2H-tetrazolum assay according to the manufacturer's instruction (Promega, Madison, Wis.). At each time point of analysis, braided nanofibrous structures were incubated in 800 μL, phenol red-free RPMI 1640 with 160 μL, MTS for 1 hour. A 200 μL, aliquot of the incubation medium was transferred to a 96-well plate and the absorbance at 492 nm was measured using a plate reader. FIG. 7

### Scanning Electron Microscopy

Braided nanofibrous structures with (i.e., cellular braided nanofibrous structures) and without cells were sputter coated with platinum and imaged using a FESEM (LEO 1530, Zeiss, Germany) at an acceleration voltage of 3 kV. Before sputter coating, cellular braided nanofibrous structures that were seeded with human mesenchymal stem cells and cultured for 7 days were collected, fixed in 2.5% glutaraldehyde for 2 hours, dehydrated using a series of graded concentrations of ethanol, and dried using CO₂ critical point drying.

FIGS. 2A (10,000×) and 2B (5,000×) are scanning electron micrographs of nanofibers prepared using PLLA. The nanofiber bundles were composed of nanofibers oriented in a uniaxial direction (scale bar 2 μm). The nanofiber bundles were then braided together into braided nanofibrous structures containing 3, 4, or 5 nanofiber bundles using different braiding patterns. See, FIG. 4.

It was discovered that increasing the number of nanofiber bundles enhanced the mechanical properties of the braided nanofibrous structures. FIG. 6 shows the tensile load-displacement curves braided nanofibrous structures made using PLLA with 3, 4, or 5 nanofiber bundles. Maximum tensile strain, maximum tensile strength, and stiffness all increased with nanofiber bundle number (Table 1). A range of mechanical properties was achieved by varying the number of nanofiber bundles from 3 to 5. For example, maximum tensile strain ranged from about 11.3 to about 24.1%. Maximum tensile strength ranged from about 6.9 N to about 12.9 N. Stiffness ranged from about 2.31 N/mm to about 3.36 N/mm.

Cell proliferation assays demonstrated that human mesenchymal stem cells cultured in the braided nanofibrous structures increased from 7 to 21 days. See, FIG. 7. Thus, braided nanofibrous structures support cell proliferation. Additionally, these results demonstrate that the post-fabrication braiding process did not affect cell growth. Analysis by scanning electron microscopy for cell morphology and adhesion further demonstrated that cells surrounded individual nanofibers and formed cell sheets spanning multiple adjacent nanofibers. See, FIG. 8.

As shown in the Example, braided nanofibrous structures prepared by braiding nanofiber bundles of individual nanofibers oriented in a uniaxial direction enhanced mechanical properties such as strain, tensile strength, and stiffness. Cell proliferation results also demonstrated that human mesenchymal stem cells cultured with braided nanofibrous structures increased during the culture period. Further, SEM of braided nanofibrous structures seeded with human mesenchymal stem cells and cultured for 7 days revealed cell adhesion to individual fibers, elongated cell morphology and the formation of cell sheets that spanned multiple adjacent fibers.

What is claimed is:

1. A braided nanofibrous structure comprising:
at least three nanofiber bundles, wherein the nanofiber bundles are braided together and each nanofiber bundle independently comprises at least two nanofibers oriented in a uniaxial direction.

2. The braided nanofibrous structure as set forth in claim 1, wherein the nanofibers are about 2 μm or less in diameter.

3. The braided nanofibrous structure as set forth in claim 1, further comprising a plurality of cells.

4. The braided nanofibrous structure as set forth in claim 1, comprising three nanofiber bundles.

5. The braided nanofibrous structure as set forth in claim 1, comprising four nanofiber bundles.

6. The braided nanofibrous structure as set forth in claim 1, comprising five nanofiber bundles.

7. The braided nanofibrous structure as set forth in claim 3, wherein the plurality of cells are selected from the group consisting of ligament fibroblasts, tenocytes, muscle fibroblasts, muscle cells, mesenchymal stem cells, embryonic stem cells, and combinations thereof.

8. The braided nanofibrous structure as set forth in claim 7, wherein the plurality of cells are selected from the group consisting of autologous cells, allogeneic cells, and xenogeneic cells,

9. The braided nanofibrous structure as set forth in claim 1, wherein the nanofibrous polymer comprises at least one of a natural protein and a synthetic material.

10. The braided nanofibrous structure as set forth in claim 9, wherein the textile based material comprises poly(glycolide) (PGA), poly(L-lactic acid) (PLA), poly(lactide-co-glycolide) (PLGA), poly(L-lactide) (PLLA), poly(D,L-lactide) (P(DLLA)), poly(ethylene glycol) (PEG), poly(ε-caprolactone) (PCL), montmorillonite (MMT), poly(L-lactide-co-ε-caprolactone) (P(LLA-CL)), poly(ε-caprolactone-co-ethyl ethylene phosphate) (P(CL-EEP)), poly[bis(p-methylphenoxy)phosphazene] (PNmPh), poly(3-hydroxybutyrate-co-3-hydroxyvalerate) (PHBV), poly(ester urethane) urea (PEUU), poly(p-dioxanone) (PPDO), polyurethane (PU), polyethylene terephthalate (PET), poly(ethylene-co-vinylacetate) (PEVA), poly(ethylene oxide) (PEO), poly(phosphazene), poly(ethylene-co-vinyl alcohol), and combinations thereof.

11. The braided nanofibrous structure as set forth in claim 1, wherein the nanofiber bundle is porous.

12. The braided nanofibrous structure as set forth in claim 11, wherein the nanofiber bundle comprises a porosity of from about 10% to about 95%.

13. The braided nanofibrous structure as set forth in claim 11, wherein the nanofiber bundle comprises pores having a pore size of from about 10 μm to about 800 μm.

14. A method of preparing a braided nanofibrous structure, the method comprising:
preparing a nanofiber bundle comprising at least two nanofibers, wherein each nanofiber bundle independently comprises at least two nanofibers oriented in a uniaxial direction; and
preparing a braided nanofibrous structure by braiding at least three nanofiber bundles together.

15. The method as set forth in claim 14, further comprising:
seeding the braided nanofibrous structure with a plurality of cells to form a braided nanofibrous structure having cells dispersed therein; and
culturing the braided nanofibrous structure having cells dispersed therein in a bioreactor.

16. The method as set forth in claim 14, wherein the nanofibers are about 2 μm or less in diameter.

17. The method as set forth in claim 14, further comprising coating the braided nanofibrous structure with at least one of a cell adhesion molecule or a bioactive molecule.

18. The method as set forth in claim 15, wherein the plurality of cells comprise ligament fibroblasts, tenocytes, muscle fibroblasts, muscle cells, mesenchymal stem cells, embryonic stem cells, and combinations thereof.

19. The method as set forth in claim 15, wherein the culturing the braided nanofibrous structure seeded with the plurality of cells further occurs in the presence of a growth factor.

20. The method as set forth in claim 19 wherein the growth factor is selected from at least one of growth differentiation factors, epidermal growth factors, basic fibroblast growth factors, and transforming growth factor-beta.
